(19) United States
(12) Patent Application Publication
(10) Pub. No.: US 2006/0211632 A1
(43) Pub. Date: Sep. 21, 2006

(54) PXR agonists for cardiovascular disease

(76) Inventors: Kenneth A. Bachmann, Sylvania, OH (US); James T. Slama, Toledo, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 11/084,209

(22) Filed: Mar. 17, 2005

(51) Int. Cl. A61K 31/7024; A61K 31/7048; A61K 31/57; A61K 31/4745; A61K 31/56; A61K 31/55; A61K 31/455; A61K 31/366; A61K 31/4166; A61K 31/337

(52) U.S. Cl. 514/23; 514/171; 514/27; 514/383; 514/456; 514/548; 514/460; 514/217; 514/291; 514/680; 514/389; 514/355; 514/449

(57) ABSTRACT

A method of treating or preventing coronary artery disease in an animal includes increasing blood serum apoA1 level in the animal by administering an effective amount of an agonist of the orphan nuclear receptor PXR. A method of identifying a substance useful for treating or preventing coronary artery disease in an animal by increasing blood serum apoA1 level in the animal, includes determining whether the substance is an agonist of the orphan nuclear receptor PXR. A composition for treating or preventing coronary artery disease in an animal by increasing blood serum apoA1 levels in the animal includes an effective amount of a combination of an agonist of the orphan nuclear receptor PXR and an agonist of the orphan nuclear receptor PPARα.

### BACKGROUND OF THE INVENTION

This invention relates in general to substances that are useful for the prevention or treatment of heart disease. More specifically, the invention relates to substances that achieve this benefit by stimulation (agonism) of a particular orphan nuclear receptor.

The Framingham heart study initiated in 1948 uncovered an inverse correlation between HDL cholesterol (HDL-C) and risk for coronary artery disease (CAD) in men and women. Other studies and publications have also linked raising HDL-C to a decreased risk of CAD. For example, the Dec. 21, 2000 supplement to the American Journal of Cardiology was entitled, “The Imperative to Raise Low Levels of High Density Lipoprotein Cholesterol—A Better Clinical Strategy in the Prevention of Coronary Artery Disease.”

Most recently, the administration of a recombinant apolipoprotein A1 (apoA1) known as apoA1 Milano has been shown to reverse atherosclerotic lesions in humans. (Nissen et al., “Effect of recombinant apoA-1 Milano on coronary atherosclerosis in patients with acute coronary syndromes,” JAMA 2003; 290: 2292-2300.) ApoA1 is the major apolipoprotein within HDL-C particles.

U.S. Pat. No. 6,103,733 to Bachmann et al. discloses a method of increasing HDL cholesterol levels by the selective induction of hepatic cytochrome P450IIIA (CYP3A) activity. Heteroaromatic phenylmethanes having a certain structural formula are described as being particularly effective. Among the disclosed compounds are clotrimazole (structure III), CDD 3540 (structure XXXXI), and CDD 3538 (structure XXXIX).

Nuclear receptors are molecules (transcription factors) that are activated by specific ligands and directly regulate the expression of target genes. These receptors are involved in controlling a wide variety of physiological processes, many of which are implicated in disease. Because nuclear receptors can provide a direct link between a ligand (such as a hormone or drug) and a physiological process, these molecules are attractive targets for drug discovery. In the 1990s, a new class of nuclear receptors was discovered which, because they had no known ligands, were called “orphan” nuclear receptors (ONRs).

The various roles of orphan nuclear receptors in lipid metabolism are becoming increasingly appreciated. PPARα (peroxisome proliferator-activated receptor-α) stimulation by drugs such as the fibrates may subsequently upregulate SR-B1 (scavenger receptor class B type 1), ABCA-1, LPL (lipoprotein lipase), ApoA1, and ApoA2 (apolipoprotein A2) genes. (Fruchart, “Peroxisome proliferator-activated receptor-α activation and high-density lipoprotein metabolism,” Am J Cardiol 2001; 88 (suppl): 24N-29N.) Activation of LXR (liver X receptor) increases the expression of ABCA1 (ATP binding cassette transporter A1) and other genes in macrophages promoting cholesterol efflux from macrophages to HDL particles. (Repa et al., “Regulation of absorption and ABC1-mediated efflux of cholesterol by RXR heterodimers,” Science 2000; 289: 1524-1529.) FXRs (farnesoid X receptors) repress bile acid synthesis, and FXR null mice exhibit elevated serum triglyceride and cholesterol levels. (Lu et al., “Orphan nuclear receptors as elixirs and fixers of sterol metabolism,” J Biol Chem 2001; 276: 37735-37738.) The expression of the phospholipid transfer protein gene was shown to be modulated by FXR, thus implicating FXR in HDL formation. (Urizar et al., “The farnesoid X-activated receptor mediates bile acid activation of phospholipid transfer protein gene expression,” J Biol Chem 2000; 275: 39313-39317.) RORα₁ (retinoic acid receptor-related orphan receptor) overexpression in Caco-2 cells increased rat apoA1 gene transcription, and intestinal apoA1 mRNA levels were lower in staggerer mice with an ROR gene deletion, than in wild type mice. (Vu-Dac et al., “Transcriptional regulation of apolipoprotein A-I gene expression by the nuclear receptor RORα,” J Biol Chem 1997; 272: 22401-4.) The PXR (pregnane X receptor) has been shown to play a role in the transport of bile acids (Staudinger et al., “Coordinate regulation of xenobiotic and bile acid homeostasis by pregnane X receptor,” Drug Metab Dispo 2001; 29: 1467-1472) and in their detoxification (Xie et al., “An essential role for nuclear receptors SXR/PXR in detoxification of cholestatic bile acids,” Proceedings National Acad Sci 2001; 98: 3375-3380).

### SUMMARY OF THE INVENTION

This invention relates to a method of treating or preventing coronary artery disease in an animal which comprises increasing blood serum apoA1 level in the animal by administering an effective amount of an agonist of the orphan nuclear receptor PXR.

In a particular embodiment, the invention relates to a method of treating or preventing coronary artery disease in an animal which comprises increasing blood serum apoA1 level in the animal by administering an effective amount of an agonist of the orphan nuclear receptor PXR, the agonist selected from the group consisting of CDD 3543, rifampin, hyperforin, topiramate, carbamazepine, dexamethasone, lovastatin, nifedipine, paclitaxel, phenyloin, spironolactone, triacetyloleandomycin, ecteinascidin, troglitazone, targretin, progesterone, rutin, pregnenolone, metyrapone, 17-alpha-hydroxy-progesterone, estradiol, and combinations thereof.

The invention also relates to a method of identifying a substance useful for treating or preventing coronary artery disease in an animal by increasing blood serum apoA1 level in the animal, by determining whether the substance is an agonist of the orphan nuclear receptor PXR.

The invention also relates to a receptor site comprising an orphan nuclear receptor PXR that when subjected to agonism is effective to treat or prevent coronary artery disease in an animal by causing an increase in blood serum apoA1 level in the animal.

The invention also relates to a pharmaceutical composition for treating or preventing coronary artery disease in an animal by increasing blood serum apoA1 level in the animal which comprises a pharmaceutically acceptable carrier and an effective amount of an agonist of the orphan nuclear receptor PXR.

The invention also relates to a method of treating or preventing coronary artery disease in an animal which comprises increasing blood serum apoA1 level in the animal by administering an effective amount of an agonist of the orphan nuclear receptor PXR except where the agonist is clotrimazole, CDD 3540, CDD 3538, or phenobarbital.

The invention further relates to a composition for treating or preventing coronary artery disease in an animal by increasing blood serum apoA1 level in the animal which comprises an effective amount of a combination of an agonist of the orphan nuclear receptor PXR and an agonist of the orphan nuclear receptor PPARα.

Various advantages of this invention will become apparent to those skilled in the art from the following detailed description of the preferred embodiments, when read in light of the accompanying drawings.

### BRIEF DESCRIPTION OF THE DRAWINGS

FIG. 1 shows the structures of substituted tritylimidazoles that were used in experiments to determine the effects on serum HDL-C and serum apoA1 of substances that are agonists of PXR.

FIG. 2 shows correlations discovered in the experiments between increases in hepatic apoA1 mRNA and in vitro hepatic microsomal EDM activity (closed symbols), and increases in serum HDL-C and in vitro hepatic microsomal EDM activity (open symbols).

FIG. 3 shows a correlation between drug-induced increases in serum HDL-C and in vivo CYP3A activity.

FIG. 4 shows a correlation between drug-induced increases in hepatic apoA1 mRNA and in vivo CYP3A activity.

FIG. 5 shows the effects of PXR and other orphan nuclear receptor agonists on HDL-C and apoA1 levels in wild type mice.

FIG. 6 shows the effects of PXR and other orphan nuclear receptor agonists on HDL-C and apo A1 levels for PXR-KO mice.

FIG. 7 illustrates the mapping of human PXR (hPXR) agonists and representative imidazoles to the hPXR pharmacophore.

### DETAILED DESCRIPTION OF THE PREFERRED EMBODIMENTS

We have found that substances which are agonists (stimulants) of the orphan nuclear receptor PXR (pregnane X receptor) are very effective in increasing apolipoprotein A1 (apoA1), and that the PXR agonists can be used as a treatment strategy for raising apoA1 in the management and/or prevention of heart disease.

Thus, the invention provides a method of treating or preventing coronary artery disease in an animal which comprises increasing blood serum apoA1 level in the animal by administering an effective amount of an agonist of the orphan nuclear receptor PXR. In a preferred embodiment, the animal is human. The agonist can be administered in most any suitable manner as well known in the art. A pharmaceutical composition can be prepared from the agonist, or a pharmaceutically acceptable salt thereof, of a pharmaceutically acceptable level of purity excluding normal pharmaceutical additives such as dilutents and carriers. The precise dosage to be employed depends on several factors including the potency of the PXR agonist, the particular host, the severity of the condition being treated, the mode of administration and the like. In one embodiment, the composition is a pharmaceutical composition comprising a pharmaceutically acceptable carrier and the PXR agonist.

The PXR agonist can also be administered in the form of a dietary supplement or a liquid or solid food. The dietary supplement can have any suitable form. For example, it can contain the agonist admixed with the usual excipients known in the art to make tablets, capsules, soft-gel capsules or like delivery vehicles. The excipent may or may not have nutritive value (for example it may include sugars and starch), and the tablets, capsules, soft-gel capsules or like delivery vehicles may also contain vitamins, minerals or other known nutraceutical products. Any suitable liquid or solid foods, such as shakes or bars, can be used to administer the agonist. The shakes, bars or other food products contain one or more conventional ingredients having nutritional and/or caloric value, such as sugars, syrups, chocolate, cocoa powder, natural or artificial flavors such as chocolate, vanilla or other flavors, lecithin, fats or oils, and/or proteins.

Preferably, the substance used in the treatment is a selective PXR agonist, having substantially no agonist effect on other nuclear receptors besides PXR.

Also preferably, the PXR agonist is sufficiently efficacious such that the treatment method is effective to increase the blood serum level of apoA1 by at least about 40%, more preferably at least about 100%, more preferably at least about 250%, and most preferably at least about 350%.

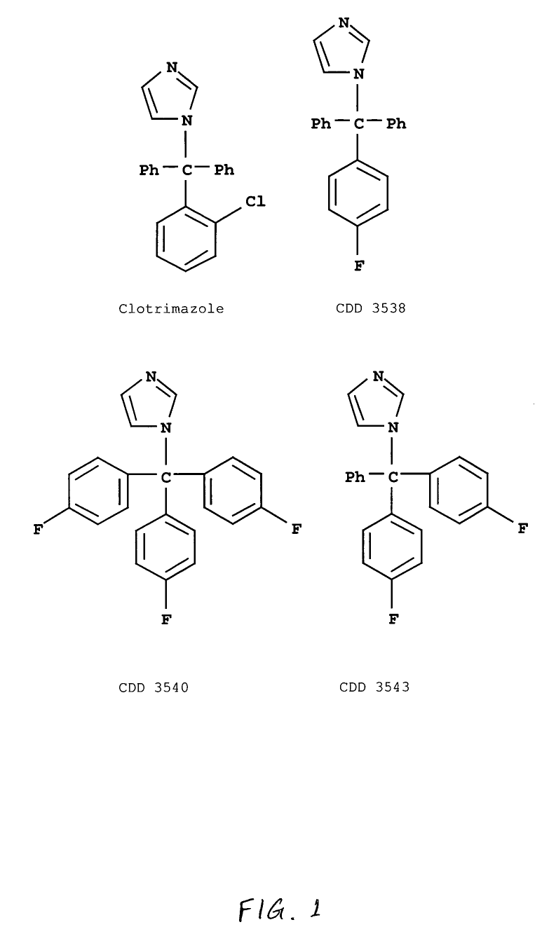

Any suitable PXR agonist can be used in the invention. In one embodiment, the agonist is selected from the group consisting of CDD 3543, rifampin, hyperforin, topiramate, carbamazepine, dexamethasone, lovastatin, nifedipine, paclitaxel, phenyloin, spironolactone, triacetyloleandomycin, ecteinascidin, troglitazone, targretin, progesterone, rutin, pregnenolone, metyrapone, 17-alpha-hydroxyprogesterone, estradiol, and combinations thereof. Some examples of other PXR agonists that can be used in the invention include CDD 3540, CDD 3538 and clotrimazole. FIG. 1 shows the structures of clotrimazole, CDD 3538, CDD 3540 and CDD 3543. The PXR agonists are typically small molecules.

In one embodiment, the invention relates to a method of treating or preventing coronary artery disease in an animal which comprises increasing blood apoA1 level in the animal by administering an effective amount of an agonist of the orphan nuclear receptor PXR except where the agonist is clotrimazole, CDD 3540, CDD 3538, or phenobarbital. In some embodiments, any of the heteroaromatic phenylmethanes disclosed in U.S. Pat. No. 6,103,733 (incorporated by reference herein) can be used as the PXR agonist; in other embodiments, these materials are excluded.

The invention also provides a method of identifying substances useful for treating or preventing coronary artery disease in an animal by increasing blood serum apoA1 level in the animal. The method involves screening to determine whether the substance is an agonist of the orphan nuclear receptor PXR. Any suitable method can be used for the screening, such as a reporter gene assay for PXR agonism.

The invention also relates to a receptor site comprising an orphan nuclear receptor PXR that when subjected to agonism is effective to treat or prevent coronary artery disease in an animal by causing an increase in blood serum apoA1 level in the animal. The orphan nuclear receptor PXR is described as follows in a journal article entitled “PXR, CAR and Drug Metabolism”, Wilson et al., Nature Reviews, vol. 1, pages 259-266 (April 2002): “The pregnane X receptor (PXR) was identified as a new orphan nuclear receptor in 1997 from a fragment that was found in the Washington University Mouse Expressed-Sequence Tag (EST) Database. Its name was based on the observation that high concentrations of 21-carbon steroids (also known as pregnanes) activated the receptor. PXR has since been cloned from a wide range of species, including mammals, birds and fish. PXR from each of these species, which functions as a heterodimer with the orphan retinoid X receptor (RXR), is activated by the naturally occurring progesterone metabolite 5β-pregnane-3,20-dione, and all the mammalian orthologues are expressed in the liver and intestine. The human PXR has also been reported as the steroid X receptor (SXR).” The Wilson et al. journal article provides additional information about PXR. Further information can be found in other journal articles, for example, “Key Structural Features of Ligands for Activation of Human Pregnane X Receptor”, Kobayashi et al., Drug Metabolism and Disposition, vol. 32, no. 4, pages 468-472 (2004).

The invention further provides a composition for treating or preventing coronary artery disease in an animal by increasing blood serum apoA1 level in the animal. The composition comprises an effective amount of a combination of an agonist of the orphan nuclear receptor PXR and an agonist of the orphan nuclear receptor PPARα.

We evaluated the combined effects of two drugs (CDD3540 and gemfibrozil) each elevating apoA1 in WT mice via different mechanisms with CDD3540 acting through PXR, and gemfibrozil acting through PPARα. We constructed dose-response curves for each drug separately in WT mice, and dose-response curves for a wide array of fixed-dose combinations. The resulting data were evaluated isobolographically. This approach demonstrated that the combined use of CDD3540 and gemfibrozil on apoA1 and HDL-C in WT mice is synergistic. More generally, the data suggest that any combination of a PXR agonist and a PPARα agonist will have a synergistic effect in increasing blood serum apoA1 level.

PPARs have been identified in many species, such as mouse, rat, and humans. They belong to a superfamily of steroid/thyroid nuclear hormone receptors. PPARs act on promoters of target genes as heterodimers with their obligate partner, RXR. There are three isotypes: α, β/δ and γ. PPARα (peroxisome proliferator-activated receptor-α) is mainly expressed in intestine, pancreas, liver, skeletal muscle, kidney, heart, and adrenals. Some examples of natural PPARα agonists are long-chain poly unsaturated fatty acids such as arachidonic acid. Some examples of synthetic PPARα agonists are fibrates (such as gemfibrozil and fenofibrate) and non-steroidal anti-inflammatory drugs.

### EXPERIMENTS

The following experiments were performed to determine the effects on serum HDL-C and serum apoA1 of substances that are agonists of PXR.

1. Materials and Methods

1.1 Materials

Clotrimazole, hyperforin, 1,4-bis[2-(3,5-dichloropyridyloxy)]benzene (TCPOBOP), rifampicin (rifampin), gemfibrozil, methylcellulose and monobasic 1.0 M potassium phosphate buffer were purchased from Sigma-Aldrich (St. Louis, Mo.). Troglitazone was purchased from Cayman Chemical (Ann Arbor, Mich.). Phenobarbital was purchased from Baker Chemical (Phillipsburg, Pa.). CDD 3540 was synthesized as described in Slama et al., “Influence of some novel N-substituted azoles and pyridines on rat hepatic CYP3A activity,” Biochem Pharmacol 1998; 55: 1881-1892. DNA oligonucleotides for genotyping PXR-KO mice were purchased from Quiagen (San Diego, Calif.).

Liquid direct HDL-C assay kits were purchased from Amresco-Inc. (Solon, Ohio). Lipi+plus (direct lipid control sets) were purchased from Polymedco, Inc. (Cortland Manor, N.Y.). Affinity Purified Anti-Mouse ApoLipoprotein A-1 (Goat) was purchased from Rockland-Inc. (Gilbertsville, Pa.), and Purified Mouse ApoLipoprotein A-1 (ApoA1) was purchased from Biodesign International (Saco, Me.).

1.2 Animals

Male Sprague-Dawley rats weighing between 140 and 270 g were used in groups of 3-6. All rats had free access to standard chow and water. The rats were kept in the vivarium at 24° C. on a 12-hour light and dark cycle. Animals were acquired from Harlan Sprague Dawley, Inc., Indianapolis, Ind.

Male wild-type (WT; C57BL/6) mice weighing 20-30 g were also purchased from Harlan Sprague Dawley. PXR knockout (PXR-KO) breeders were generously provided by Dr. Ronald M. Evans at the Salk Institute, La Jolla, Calif., and a colony of PXR-KO mice was raised and maintained at the central animal facility of the University of Toledo, Toledo, Ohio.

1.3 Treatment of Rats

Rats were treated in groups of 3-6 with either a methylcellulose suspension (1.0%), or with clotrimazole, or with one of the CDD compounds suspended in 1.0% methylcellulose as the vehicle. (FIG. 1 shows the structures of clotrimazole and the CDD compounds.) Doses were 100 mg/Kg unless otherwise specified. Treatments were administered by gavage once daily in the morning for eight (8) days.

Ethosuximide was prepared as a 3.5% (w/v) solution dissolved in normal saline. Ethosuximide was administered in a dose of 35 mg/kg via tail vein injection 24 hours following the eighth pretreatment dose.

1.4 Treatment of Mice

WT mice were treated with one of these eight chemicals: clotrimazole (CTZ), troglitazone, hyperforin, rifampicin, CDD 3540 (FIG. 1), gemfibrozil, phenobarbital or TCPOBOP. PXR-KO mice were treated with either rifampicin, gemfibrozil, or CDD 3540. In all cases methylcellulose was used as a vehicle control. These chemicals were administered, as a suspension in 1% methylcellulose (vehicle) by gavage using a 20 G—1½-inch feeding needle. The animals were treated in groups of three and for each treated group two mice were treated simultaneously with methylcellulose (vehicle). Each treatment was given once daily in the morning for seven days. The doses were 100 mg/kg for CTZ, rifampicin, CDD 3540, phenobarbital and TCPOBOP, 6 mg/kg and 60 mg/Kg for troglitazone and 0.175 mg/Kg and 1.75 mg/Kg for hyperforin, and for gemfibrozil 20 mg/Kg and 100 mg/Kg. The volume of vehicle used to suspend the treatment drugs and the dose of vehicle for control group was 7 ml/kg. Doses were selected based on the results obtained from previous studies in rats except for troglitazone and hyperforin, for which doses were weight-adjusted using estimates of average daily human doses as reference.

1.5 Genotyping of PXR-KO Mice

DNA was prepared by adding 600 μL of 50 mM NaOH to tail biopsies, and then boiled at 100° C. for 10 minutes. 50 μL of 1 M Tris-HCl, pH 8.0 was added, and 1 μL of the final solution was used for PCR reactions.

Genotyping was performed using a 3-primer PCR assay that produces alternate products from WT and PXR-KOs. A standard PCR mixture (10 μL) was added to 1 μL of the final DNA preparation. The PCR program was as follows: The initial PCR was run at 95° C. for 1 min. Subsequently programs of 95° C. for 15 sec, 55° C. for 30 sec, and 72° C. for 1 min were run with the sequence repeated forty times.

PCR products were resolved by electrophoresis through a 3% agarose gel. Oligonucleotide sequences were as follows:

| | |
|---|---|
| WX48: | AGA AAC ACA TAG AAA CCC CAT G |
| WX47: | AGT CCA CCA AGC CTG AGC CTC C |
| WXNEO: | CTT GAC GAG TTC TTC TGA GGG GAT C |

The mutant allele produces a band at 546 bp, and the WT allele produces a band at 497 bp.
1.6 Samples Collected from Rats

Whole blood was collected by cardiac puncture under CO2 anesthesia 8 hours following ethosuximide infusion. Animals were then sacrificed using CO2 and cervical dislocation. Livers were excised and used for analysis of erythromycin demethylase activity (as described in the Slama et al. reference) and hepatic apoA1 mRNA. Plasma was separated from whole blood anticoagulated with 5 mM EDTA at room temperature using a bench top centrifuge (5000 rpm) for 5 minutes. Plasma was stored unpreserved at −20° C. for up to 5 days, and subsequently analyzed for ethosuximide and for HDL-C. Concentrations of ethosuximide and HDL-C were measured in plasma by fluorescence polarization immunoassay (fpia) using a TDx analyzer (Abbott Laboratories, Irving, Tex.) and commercially available reagents (Abbott Laboratories, Abbott Park, Ill.). The assays exhibited a coefficient of variation of <5% and detection limits of 0.5 mg/l and 2 mg/dL, respectively.

Hepatic apoA1 mRNA was measured by Northern Blot hybridization to total hepatic RNA with oligonucleotides for apoprotein A1 and elongation factor-1α (EF-1α). The procedure was similar to that described in the Slama et al. reference for hepatic CYP3A mRNA. The following oligonucleotide sequence used for rat apoA1 mRNA was based on the nucleotide sequence for rat apoA1 described by Haddad et al. (“Linkage, evolution and expression of the rat apolipoprotein A-1, C-III, and A-IV genes,” J Biol Chem 1986; 261: 13268-13277): 3′ CTA CGT CAG TTC CTG TCG CCG TCT CTG ATA CAC AGG GTC AAA CTT AGG AGG TGA AAC CCG 5′.

The time course for changes in apoA1 mRNA was evaluated only in rats treated with CDD 3540, an analog of clotrimazole in which the o-chlorine atom is replaced with hydrogen, and a p-substituted fluorine added to each aromatic ring (FIG. 1). Twenty-four rats were dosed with CDD 3540 (50 mg/Kg) by gavage once daily for 8 days, and three rats were treated with methylcellulose (vehicle controls). Groups of three treated rats were sacrificed at 6 and 12 h after the first dose, 12 and 24 h after the second dose, and at 12 hours after doses 4-8, respectively. Hepatic apoA1 mRNA was quantitated by northern blot analysis as described above.

1.7 Samples Collected from Mice

After the completion of the treatment (i.e. on day 8), blood samples were collected from the tail of the animals for serum HDL-C and ApoA1 determination. Blood samples were allowed to clot for 2 hours, centrifuged at 14,000 rpm for 20 minutes, and serum was collected. The serum samples were then stored at −20° C. for up to 24 hours before quantitative determination of HDL-C and ApoA1.

HDL-C was determined quantitatively in the serum using Liquid Direct HDL kits. The kit contained reagent 1, reagent 2, and HDL calibrator. The assay consisted of two steps. In the first step, 300 μL of reagent 1, a solution containing a buffer (pH 7.0), cholesterol esterase (yeast), cholesterol oxidase (bacteria), catalase (bovine liver), ascorbate oxidase (bacteria), N-(2-Hydroxy-3-sulfopropyl)-3,5-dimethoxyaniline, sodium salt (HDAOS) and stabilizers, was added to 4 μL of serum. This results in the removal of lipoproteins other than HDL (i.e. LDL, VLDL & chylomicrons) via selective reaction with cholesterol esterase and cholesterol oxidase. The peroxide byproduct of this reaction is rendered non-colored via catalase reduction. In the second step, 100 μL of reagent 2, a solution containing a buffer (pH 7.0), peroxidase (horseradish), 4-aminoantipyrine, surfactants and 0.05% sodium azide, was added to the above mixture after 5 minutes, and the absorbance of the resultant mixture was observed at 600 nm after 3 minutes using a Beckmann DU640 Spectrophotometer. In this second step, the catalase is inhibited, the remaining HDL cholesterol specifically reacts with cholesterol esterase and cholesterol oxidase, and in the presence of peroxidase the peroxide byproduct reacts with 4-aminoantipyrine and HDAOS to form a colored quinine dye. Both these reactions were allowed to occur at 37° C. A calibrator provided with the kit was used as a reference to calculate the concentration of the samples as follows:

$$Concentration of Sample = \frac{Concentration of Calibrator \times Sample Absorbance}{Calibrator Absorbance}$$

Two quality control standards were used used to confirm the accuracy of the assay procedure with nominal HDL-C concentrations of 40.1 to 58.1 mg/dL and 74.7 to 88.2 mg/dL, respectively. Each sample was analyzed twice, and the average of both readings was used.

An immuno-turbidimetric assay was developed for quantitative determination of ApoA1 in mouse serum. Commercially available stock solution of purified mouse ApoA1 (79 mg/dl) was used to construct a standard curve. A potassium phosphate buffer solution was prepared by diluting 1 ml of monobasic 1.0 M potassium phosphate buffer to 10 ml with distilled water, and the pH of the solution was adjusted to 7.2 using 0.1 N NaOH. Polyclonal, affinity purified anti-mouse apoLipoprotein A-1 antibody (1.0 mg/ml) was used for this assay. A dilution of 1:4000 of the stock concentration was used per the manufacturer's recommendation. The standard curve was prepared with 0, 39.5, 79, and 158 mg/dl of mouse apoA1. 1 μl of mouse plasma was mixed with 3 μl of distilled water for quantitation of apoA1. 500 μl of buffer solution was added to 4 μl of total sample volume prepared as described. After 5 minutes, 100 μl of antibody solution was added. The mixture was allowed to incubate at 37° C. for 3 minutes. Absorbance was measured at 580 nm using a Beckmann DU640 Spectrophotometer. Each sample or standard was analyzed twice, and the average of both readings was used. Serum ApoA1 concentrations from control mice were also compared with the normal ApoA1 levels in mice, obtained from the literature, to affirm the validity of the assay procedure.

1.8 Mapping to hPXR Pharmacophore

Three imidazoles, CDD 3538, 3540, and 3543 (see FIG. 1 for structures) were sketched in Catalyst™, and multiple conformers were generated (up to 255 with an energy cutoff of 20 kcal/mol). The molecules were then mapped to the hPXR pharmacophore generated as described previously (Ekins et al., “A pharmacophore for human pregnane-X-receptor ligands,” Drug Metab Dispos 2002; 30: 96-99) but using Catalyst™ version 4.7 (Accelrys, San Diego, Calif.) (Ekins et al., “A ligand-based approach to understanding selectivity of nuclear hormone receptors PXR, CAR, FXR, LXRa and LXRb,” Pharm Res 2002; 19: 1788-1800). The mapping of the imidazoles was compared to clotrimazole and hyperforin, both known ligands for hPXR. The Daylight ClogP was also calculated in Cerius2 version 4.8 (Accelrys, San Diego, Calif.).

1.9 Statistical Analyses

Temporal differences in hepatic apoA1 mRNA in rats were evaluated by a simple Analysis of Variance (ANOVA) and a Tukey's post-hoc test.

Statistical treatments for HDL-C and apoA1 in mice were identical. For each of these variables, three basic analyses were performed, one for WT mice, one for PXR-KOs, and one for the two groups combined. Statistical analysis was performed in SAS (SAS Release 8, 1999, SAS Institute Inc., Cary, N.C.). In all cases the primary method of comparing treatments was ANOVA. Prior to performing this analysis, both variables were normalized by the geometric mean of their associated controls. Normality and homoscedasticity (constancy of variance across groups) were evaluated, with an appropriate data transformation (logarithmic) applied if necessary. These properties were checked before (to evaluate the need for transformation) and after transformation (to confirm the adequacy of the transformation) using normal probability plots and Levene's test for equality of variances (Snedecor et al., Statistical Methods, 8th Edition, Iowa State University Press, pp. 59-62 and 252-253, 1989). In the one-factor tests for WT and PXR-KOs separately, if the ANOVA null hypothesis of equality of group means was rejected, then multiple comparisons procedures for comparing treatments to control and for comparing all treatments to each other were performed. Dunnett's procedure was used for comparing treatments to control (Dunnett, “A multiple comparisons procedure for comparing several treatments with a control,” Journal of the American Statistical Association, 1955; 50: 1096-1121), while Tukey's procedure was employed for comparing all treatment pairs (Tukey, “The problem of multiple comparisons,” unpublished manuscript 1953, and Kramer, “Extension of multiple range tests to group means with unequal numbers of replications,” Biometrics 1956; 12: 307-310). For the combined analysis, a two factor ANOVA was performed to test for a statistically significant interaction between type (of mouse) and treatment (for the limited set of four treatments applied to the PXR-null mice only). Tukey's multiple comparison procedure was again used to identify type/treatment pairs that were statistically significantly different.

2. Results

2.1 Correlations Between CYP3A Activity and Measures of Hepatic apoA1 mRNA and Serum HDL-C in Rats.

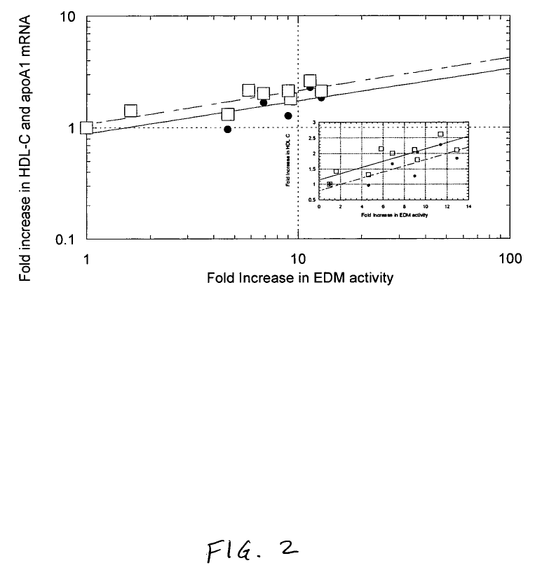

The relationships between drug-induced increases in rat hepatic apoA1 mRNA and serum HDL-C and increases in CYP3A activity are given in FIGS. 2-4. FIG. 2 depicts correlations between increases in in vitro CYP3A activity, measured as hepatic microsomal EDM (erythromycin demethylase) activity, and hepatic apoA1 mRNA (closed symbols) and serum HDL-C (open symbols), respectively. The data are plotted as the ratios of increased measures in treated animals relative to vehicle (control) animals. The regression equation is for apoA1 mRNA is y=0.883*xˆ0.291 with r=0.746. For HDL-C the equation is y=1.07*xˆ0.298 with r=0.844. Each data point depicts an experiment with a different imidazole (see Slama et al., “Influence of some novel N-substituted azoles and pyridines on rat hepatic CYP3A activity,” Biochem Pharmacol 1998; 55: 1881-1892). The inset graph depicts the same data plotted on linear axes.

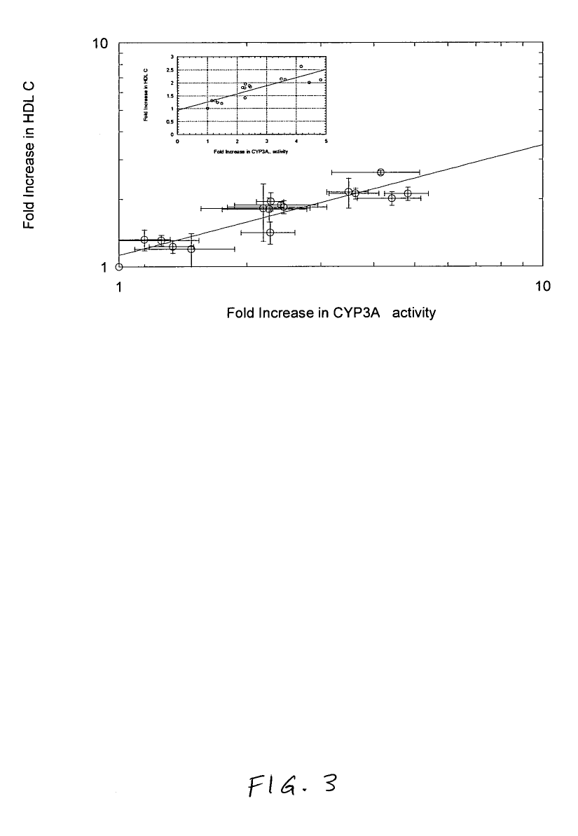

FIG. 3 correlates the increase in serum HDL-C with increases in in vivo CYP3A activity using ethosuximide clearance as a biomarker for CYP3A activity. Fold increase denotes the ratio of the measured parameter in treated animals to control (vehicle treated) animals. Each data point denotes treatment with a different imidazole. The regression equation is y=1.125*xˆ0.492, and r=0.891. Inset graph depicts the same data plotted on linear axes.

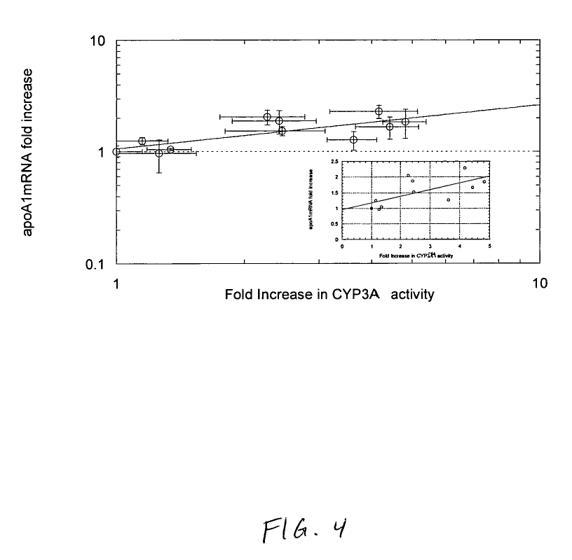

The correlation between apoA1 mRNA and CYP3A activity is depicted in FIG. 4. Fold increase denotes the ratio of the measured parameter in treated animals to control (vehicle treated) animals. Each data point denotes treatment with a different imidazole. The regression equation is y=1.06*xˆ0.394, and r=0.700. Inset graph depicts the same data plotted on linear axes.

Daily treatment with CDD 3540 (50 mg/Kg) elicited an approximate 45% increase in hepatic apoA1 in rats beginning after treatment day 2 (p<0.05 compared to vehicle controls), and remaining constant thereafter.

2.2 Treatment of WT and PXR-KO Mice with PPARα, PPARγ, CAR, and PXR Agonists.

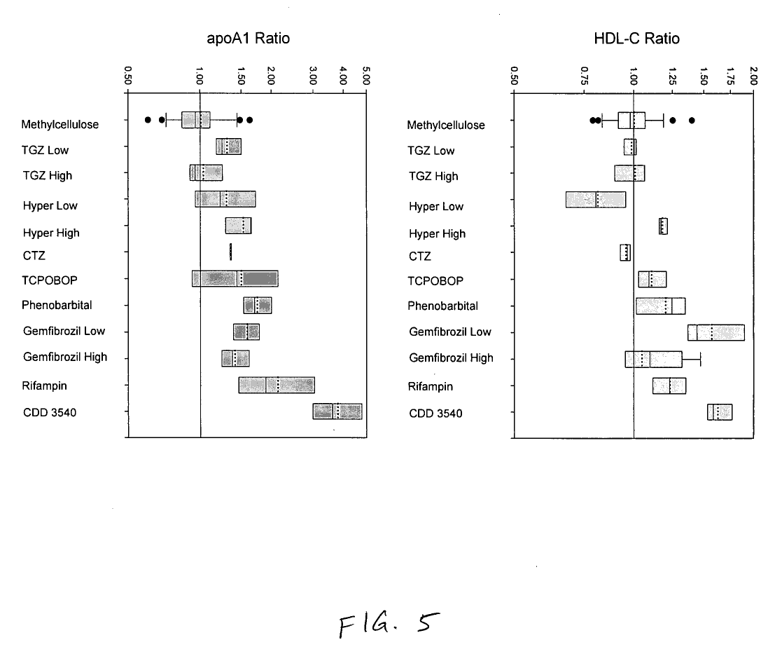

The treatment of wild-type mice with agonists for PPARα (gemfibrozil), PPARγ (troglitazone), CAR (TCPOBOP), a mixed CAR/PXR agonist (phenobarbital), and several agonists that are effective either for the human PXR (hPXR) or rodent PXR elicited results that are depicted in FIG. 5 for HDL-C and apoA1. The HDL-C and apoA1 ratios are the observed values normalized by the geometric mean of the associated controls (methylcellulose). The box plots show the mean (dotted line), median (50^{th} percentile, the solid line in the center of the box), quartiles (25^{th} and 75^{th} percentiles, the ends of each box), 10^{th} and 90^{th} percentiles (the ends of the whiskers), and outlying values. They were produced using Sigma Plot Version 8, SPSS, Inc. For datasets with fewer than nine observations, whiskers cannot be derived and are therefore not shown. Abbreviations are as follows: TGZ low and high are the low and high dose treatments of troglitazone. Hyper low and high are the low and high dose treatments with hyperforin. Gemfibrozil low and high denote the low and high dose treatments with gemfibrozil. All other doses were 100 mg/Kg.

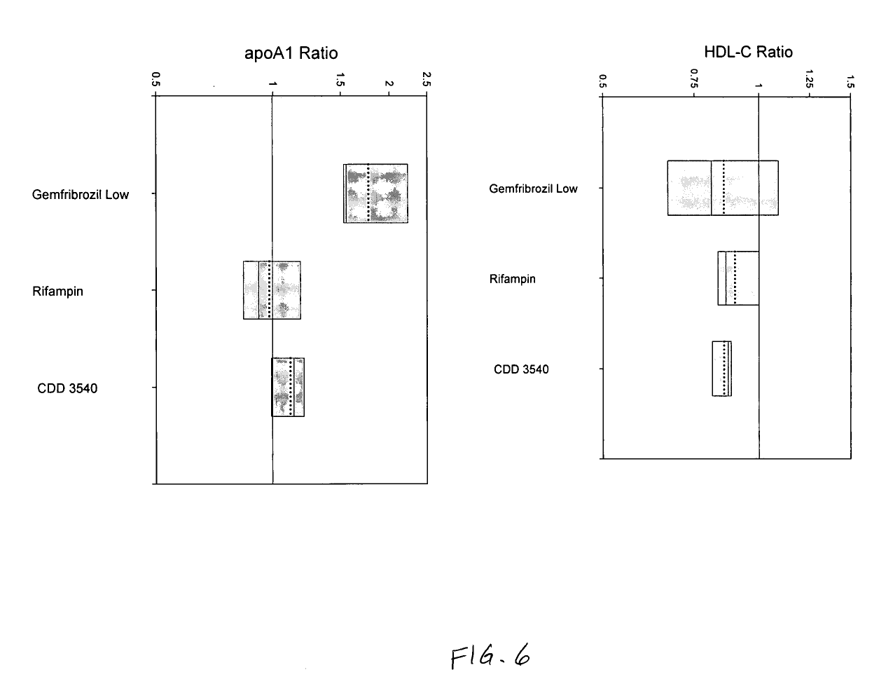

Using Dunnett's multiple comparison procedure, treatments with CDD 3540 and gemfibrozil (20 mg/kg), elicited significantly elevated levels of HDL-C relative to controls (methylcellulose). CDD 3540, rifampin, phenobarbital, and gemfibrozil (20 mg/Kg) all elicited significantly elevated levels of apoA1 relative to controls. For CDD 3540 this represented an approximate 3.7-fold increase in apoA1 relative to controls. The effects of treating PXR-KOs with either CDD 3540, gemfibrozil (20 mg/kg), or rifampin relative to the effects of control (methylcellulose) treatment on HDL-C and apoA1 levels are shown in FIG. 6. The HDL-C (left) and apoA1 (right) ratios are the observed values normalized by the geometric mean of the associate controls. These box plots show the mean (dotted line), median (50^{th} percentile, the solid line in the center of the box) and quartiles (25^{th} and 75^{th} percentiles, the ends of each box). They were produced using Sigma Plot Version 8, SPSS, Inc. Since these datasets all have fewer than nine observations, whiskers cannot be derived and are therefore not shown. For datasets with fewer than three observations, the box plots themselves cannot be created. This is the case for controls in the PXR Null mice. However, the line at Ratio=1 represents the mean of these normalized controls since they are normalized by their own geometric mean.

In the PXR-KO mice none of the treatments increased HDL-C, though gemfibrozil did significantly increase levels of apoA1 relative to control, and this approximated a 1.7-fold increase. CDD 3540 putatively acts as a PXR agonist (see below), whereas gemfibrozil is a PPARα agonist. Thus, the PPARα-related effect of gemfibrozil on apoA1 that was elicited in wild-type mice was sustained in PXR-KO mice.

2.3 Mapping Known and Putative hPXR Agonists to the hPXR Pharmacophore.

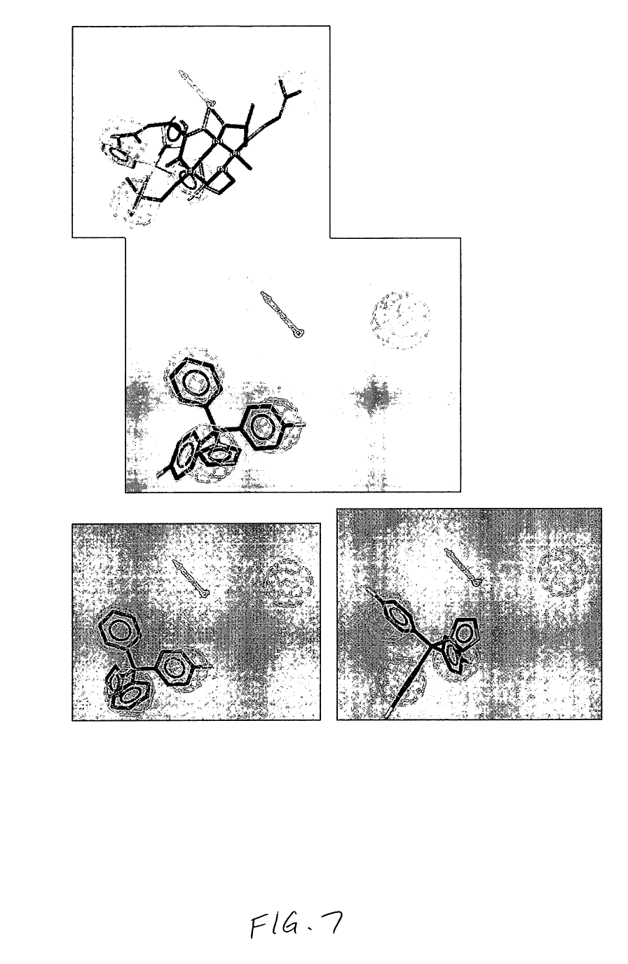

Mapping of human PXR (hPXR) agonists and representative imidazoles to the hPXR pharmacophore is depicted in FIG. 7 (cyan features=hydrophobes, green feature=hydrogen bond acceptor). At the upper left, hyperforin and clotrimazole are mapped to the hPXR pharmacophore. At the upper right, CDD 3543 is mapped to the pharmacophore, predicted EC₅₀=3.4 μM. At the lower left, CDD 3538 is mapped to the pharmacophore, predicted EC₅₀=9.6 μM. At the lower right, CDD 3540 is mapped to the pharmacophore, predicted EC₅₀=25 μM

All four analogs (CDD compounds and clotrimazole) have quite similar predicted ClogP values which is in agreement with the pharmacophore mappings, suggesting that the hydrophobic interactions are important for these molecules. The ClogP values for clotrimazole, CDD 3538, CDD 3540, and CDD 3543 are 5.2, 4.7, 5.0, and 4.8, respectively. Clearly, these imidazoles partially map to the pharmacophore with two features missed altogether. It may be possible that, due to the flexibility of the binding site, the hydrogen bond acceptor may be reached.

3. Discussion

The results show a correlation between drug-induced increases in plasma HDL-C and hepatic apoA1 mRNA, and increases in CYP3A activity measured in vitro (FIG. 2) and in vivo (FIGS. 3-4).

The potential role of PXR in apoA1 and HDL-C regulation was tested using selective orphan nuclear receptor agonists. We treated wild-type mice with a series of substances that activate various ONRs. Gemfibrozil was used as a PPARα agonist, troglitazone as a PPARγ agonist, TCPOBOP as a selective CAR agonist, phenobarbital as a mixed PXR/CAR agonist, and rifampin, clotrimazole, and CDD 3540 were used as PXR agonists. In wild-type mice gemfibrozil (20 mg/Kg) and CDD 3540 (100 mg/Kg) significantly elevated HDL-C. Rifampin (100 mg/Kg) increased HDL-C by about 25%, but that increase did not achieve statistical significance. Gemfibrozil (20 mg/Kg), CDD 3540 (100 mg/Kg), rifampin (100 mg/Kg), and even phenobarbital (100 mg/Kg) elicited significant increases in apoA1 relative to vehicle-treated control mice. Since the selective CAR agonist, TCPOBOP, did not significantly elevate either HDL-C or apoA1 levels, it is likely that the phenobarbital effects were elicited through PXR rather than CAR. The absence of effects of troglitazone is consistent with at least three different explanations: 1) troglitazone is not as good a PPARγ agonist in mice as in humans; 2) the doses of troglitazone used were too low; 3) the PPARγ receptor, while increasing cholesterol transport to apoA1 via ABCA1 up-regulation, does not play a crucial role in regulating apoA1 and/or HDL-C levels, per se.

The increased levels of apoA1 and HDL-C in wild-type mice elicited by gemfibrozil (20 mg/Kg) are consistent with a role of PPARα in their regulation. The dose-response curve for gemfibrozil reached a peak at 20 mg/Kg, and then declined with higher doses; we have not investigated the possible reasons for that phenomenon.

The centrality of the PXR in apoA1 regulation is clear when considering the data in PXR-KOs. In contrast to their effects in wild-type mice, neither rifampin nor CDD 3540 elicited significant changes in apoA1 levels relative to vehicle control mice. On the other hand, gemfibrozil significantly increased apoA1 even in the PXR-KOs. Increases in apoA1 in PXR-KOs would be expected for a substance regulating apoA1 synthesis through a non-PXR mechanism such as PPARα agonism.

We found that substituted imidazoles structurally related to clotrimazole (CDD 3538, 3540, and 3543) increase both CYP3A activity as well as apoA1 and HDL-C in rats, and that CDD 3540 increases apoA1 and HDL-C in WT mice but not in PXR-KO mice. Clotrimazole, CDD 3538, 3540, and 3543 can be partially mapped to a model of the hPXR pharmacophore. It is possible that selective hPXR agonism may produce even more profound increases in apoA1 in humans than we observed in WT mice.

In conclusion, we have established strong and positive correlations between the ability of PXR agonists to increase CYP3A activity in rats with their ability to increase apoA1 mRNA. We have further established that substances that are agonists of rodent PXR elicit dramatic increases in serum apoA1 in wild-type mice. Those same agonists of rodent PXR fail to increase serum apoA1 in PXR-KOs even though gemfibrozil, a PPARα agonist, retains the ability to increase apoA1 in PXR-KOs. We assert that collectively, these findings point to an important role for PXR or at least PXR agonists in the regulation of apoA1 in rodents, and we expect that selective hPXR agonists also play a similar role in the regulation of apoA1 in humans. These results may lead to the development of small molecules designed exclusively to elevate apoA1 for managing or preventing atherosclerosis.

In accordance with the provisions of the patent statutes, the principle and mode of operation of this invention have been explained and illustrated in its preferred embodiments. However, it must be understood that this invention may be practiced otherwise than as specifically explained and illustrated without departing from its spirit or scope.

What is claimed is:

1. A method of treating or preventing coronary artery disease in an animal which comprises increasing blood serum apoA1 level in the animal by administering an effective amount of an agonist of the orphan nuclear receptor PXR.

2. A method according to claim 1 wherein the agonist is a selective PXR agonist.

3. A method according to claim 1 which is effective to increase the apoA1 level by at least about 40%.

4. A method according to claim 1 which is effective to increase the apoA1 level by at least about 100%.

5. A method of treating or preventing coronary artery disease in an animal which comprises increasing blood serum apoA1 level in the animal by administering an effective amount of an agonist of the orphan nuclear receptor PXR, the agonist selected from the group consisting of CDD 3543, rifampin, hyperforin, topiramate, carbamazepine, dexamethasone, lovastatin, nifedipine, paclitaxel, phenyloin, spironolactone, hyperforin, triacetyloleandomycin, ecteinascidin, troglitazone, targretin, progesterone, rutin, pregnenolone, metyrapone, 17-alpha-hydroxy-progesterone, estradiol, and combinations thereof.

6. A method of identifying a substance useful for treating or preventing coronary artery disease in an animal by increasing blood serum apoA1 level in the animal, by determining whether the substance is an agonist of the orphan nuclear receptor PXR.

7. A method according to claim 6 which includes determining whether the substance is sufficiently efficacious such that administering the substance to an animal increases blood serum apoA1 level in the animal by at least about 50%.

8. A method according to claim 6 which includes determining whether the substance is a selective PXR agonist.

9. A receptor site comprising an orphan nuclear receptor PXR that when subjected to agonism is effective to treat or prevent coronary artery disease in an animal by causing an increase in blood serum apoA1 level in the animal.

10. A pharmaceutical composition for treating or preventing coronary artery disease in an animal by increasing blood serum apoA1 level in the animal which comprises a pharmaceutically acceptable carrier and an effective amount of an agonist of the orphan nuclear receptor PXR.

11. A method of treating or preventing coronary artery disease in an animal which comprises increasing blood serum apoA1 level in the animal by administering an effective amount of an agonist of the orphan nuclear receptor PXR except where the agonist is clotrimazole, CDD 3540, CDD 3538, or phenobarbital.

12. A composition for treating or preventing coronary artery disease in an animal by increasing blood serum apoA1 level in the animal which comprises an effective amount of a combination of an agonist of the orphan nuclear receptor PXR and an agonist of the orphan nuclear receptor PPARα.
